(19) United States
(12) Patent Application Publication
(10) Pub. No.: US 2026/0294405 A1
(43) Pub. Date: Oct. 1, 2026

(54) MRI-COMPATIBLE BIOPSY ROBOT

(71) Applicant: Promaxo, Inc., Oakland, CA (US)

(72) Inventors: Akram Gholami PAREH, Oakland, CA (US); Lars PETERSEN, Oakland, CA (US); Neel SANCHETI, Milpitas, CA (US); Sreeja VANGAPELLI, Oakland, CA (US); Paul BRATHWAITE, Dublin, CA (US); Aleksandar NACEV, San Francisco, CA (US); Nariman Majdi NASAB, Oakland, CA (US)

(21) Appl. No.: 19/372,085

(22) Filed: Oct. 28, 2025

Related U.S. Application Data
(63) Continuation of application No. PCT/US2024/026646, filed on Apr. 26, 2024.
(60) Provisional application No. 63/462,916, filed on Apr. 28, 2023.

(51) Int. Cl. A61B 10/02; A61B 5/055; A61B 17/00; A61B 17/34; A61B 34/20; A61B 34/32; A61B 50/13

(52) U.S. Cl. CPC A61B 10/0233; A61B 5/055; A61B 17/3403; A61B 34/32; A61B 50/13; A61B 2010/0208; A61B 2017/00911; A61B 2034/2055

(57) ABSTRACT

Disclosed herein is a robotic system that can be used for needle-guided diagnostics. The diagnostics can include biopsies. Such a robot may be configured for use with MRI devices, for example including low-field strength MRI scanners. Such a robot may be size accessible within closed-bore tunnel-shaped scanners.

### CROSS-REFERENCE

This application is a continuation of International Patent Application No. PCT/US2024/026646, filed Apr. 26, 2024, which claims the benefit of U.S. Provisional Application No. 63/462,916, filed Apr. 28, 2023, the disclosure of which is incorporated herein by reference in its entirety.

### BACKGROUND

Biopsy can be used to remove a sample from a subject for testing, for example cancer testing. Biopsy may rely on the radiologist's manual insertion of the needle. Imaging techniques such as magnetic resonance imaging (MRI) can be applied to localize lesions before the intervention.

### SUMMARY

In one aspect, a biopsy robot system is disclosed. The biopsy robot system comprises: (a) a needle driver configured to insert a biopsy needle into a subject; (b) a linear mechanism configured to place the needle driver inside a magnetic resonance scanner bore; and (c) a parallel manipulator configured to orient or position the linear mechanism.

In some embodiments, the biopsy robot system further comprises a robot cart configured to house components (a)-(c).

In some embodiments, the robot cart is configured to be attached, secured, or both to a MRI scanner.

In some embodiments, the biopsy robot system further comprises at least one optical sensor configured to be placed behind the needle driver to position the needle.

In some embodiments, the at least one optical sensor is configured to confirm an accuracy of needle cockings and firings.

In some embodiments, the parallel manipulator is fixed inside the magnetic resonance scanner bore.

In some embodiments, the parallel manipulator provides at least three degrees of freedom.

In some embodiments, the parallel manipulator is configured to connect the end-effector to a base using a plurality of parallel kinematic chains.

In some embodiments, the needle driver has at least one degree of freedom.

In some embodiments, the biopsy robot system has at least four degrees of freedom.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 illustrates an example robotic system's isometric view, in accordance with some embodiments.

FIG. 2 illustrates a top view of an example robotic system, in accordance with some embodiments.

FIG. 3 illustrates an isometric view of an example manipulator, in accordance with some embodiments.

FIG. 4 illustrates a top view of an example manipulator schematic, in accordance with some embodiments.

FIG. 5 illustrates a top view of an example manipulator and an example needle driver at a random configuration other than the home position, in accordance with some embodiments.

FIG. 6 illustrates a side view of an example needle driver at its home position (top) and as the needle may be moved forward for insertion (bottom) in accordance with embodiments described herein.

FIG. 7 illustrates a side and forward view of an example hexagonal socket end of the lead screw in the needle driver in accordance with embodiments described herein.

FIG. 8 illustrates a side view of the automatic firing mechanism, in accordance with some embodiments.

FIG. 9 illustrates an example isometric view of an example robot beside a low-field MRI system, in accordance with some embodiments.

FIG. 10 illustrates an isometric view of an example manipulator and needle driver inside a low-field MRI system and an example sample collection mechanism in front, in accordance with some embodiments.

FIG. 11 illustrates an isometric view of an example use of the present system, in accordance with some embodiments.

FIG. 12 illustrates a magnified isometric view of a view of an example system while operating a prostate biopsy procedure, in accordance with some embodiments.

FIG. 13 illustrates an isometric view of an example stand-alone configuration of an example manipulator and needle driver, in accordance with some embodiments.

FIG. 14 illustrates a side view of a stand-alone configuration of an example manipulator and needle driver, in accordance with some embodiments.

FIG. 15 illustrates a stand-alone configuration of an example sample collection mechanism attached to an example MRI scanner dish in accordance with embodiments described herein.

### DETAILED DESCRIPTION

The present disclosure relates to medical devices. More particularly, a robotic arm made of medical imaging-safe materials that can be utilized for needle-guided interventions.

Imaging techniques such as magnetic resonance imaging (MRI) can be applied to localize lesions before the intervention and to guide the needle through the procedure using imaging feedback. Biopsy can rely on the radiologist's manual insertion of the needle, and iterative positioning and imaging may be involved due to insufficient physician grasp on patients in the MR scanner bore, which may increase patient risk. In contrast, robotic approaches may add higher stiffness and precision by a more stabilized robotic manipulator than human hands. The robotic manipulator can support the retraction of the needle, including a tissue sample, more accurately. In some cases, a robust teleoperated system and instrumented testing platform can be used for robotic MRI-guided percutaneous puncture. In some cases, a semi-automated robotic system accomplished valid needle navigation with an error of 0.89±0.31 mm for MRI-guided percutaneous needle practices. In some cases, a robotic system can be used for transperineal prostate biopsy guided by MRI directly.

Image-guided biopsy robots may be a technological advancement in medical interventions that aim to improve clinical outcomes through more accurate on-target selection, less invasive, reduced procedure infection, and more effective procedures. These robots can utilize medical imaging-based feedback to guide the biopsy needle to the target tissue. Using an MRI-guided biopsy robot can reduce procedure time as patients do not need to be moved out of the bore for intervention and back into the bore for imaging due to the lack of available space for the procedure. This can eliminate the time-consuming patient repositioning process and may enable simultaneous intervention and imaging. Additionally, MRI-guided biopsy robots can improve ergonomics by reducing the challenges associated with manual interventions in a closed-bore scanner, which can be challenging and sometimes unfeasible due to spatial constraints inside the MRI bore.

Two main groups of biopsy robots may exist: systems with manually controlled needle insertion and systems with automated needle insertion. In the first group, the robot can control the position of the needle guide, and the clinician can manually insert the needle into the body. In the second group, the needle guide may be robotically positioned, and the robot may insert the needle into the body. This disclosure aims to develop a fully automated biopsy system where the robot can control the needle guidance, insertion, and multiple tissue sampling.

Due to the high cost of high tesla magnets, space requirements, and easy access to the imaging modality, there may be increasing interest in using low-field MRI scanners, which can provide diagnostically useful information even if the image quality may be not as high as that produced by high-field MRI scanners. The disclosed system may provide a new robot for MRI-guided interventions compatible with different types of MRI scanners, especially low-field scanners with small bores.

In summary, image-guided biopsy robots may offer several advantages, including more accurate and less invasive procedures, reduced procedure time, improved ergonomics, and improved access to medical interventions for patients in developing countries. Developing a fully automated biopsy procedure and a robot compatible with different MRI scanners may be a promising solution in biopsy interventions.

The present disclosure features a robotic system that can be used for needle-guided diagnostics. Such medical devices can include, but may be not limited to, percutaneous interventions such as biopsies or thermotherapy. In some embodiments, such a robot may be configured for use with MRI devices, for example including low-field strength MRI scanners. Such a robot may be size accessible within closed-bore tunnel-shaped scanners. The robotic system can collect and store multiple samples. The robot may be constructed of materials compatible with the working environment associated with MRI, such as non-magnetic and dielectric materials such as plastics (e.g., thermoplastic polymers, such as polyvinyl chloride (PVC) or polyethylene terephthalate (PET)), ceramics, rubbers, or any combination thereof. The present robot can be used in at least two different configurations. In the first configuration, the robot may contain a cart with a linear guide that moves the robot inside the MRI scanner bore. The robot can be fixed inside the MRI bore in the second configuration.

To ensure accuracy and effectiveness in biopsy procedures, configuring the structure and workspace of a biopsy robot may be important. Serial manipulators can offer a larger workspace and more degrees of freedom, but designing them for stiffness and strength can be challenging and may impact biopsy accuracy. In contrast, parallel manipulators may be often preferred for biopsy robots due to their precise positioning and ability to be designed with high stiffness and accuracy. This may be because parallel manipulators utilize multiple parallel links to connect the end effector to the base, thereby increasing stiffness and accuracy.

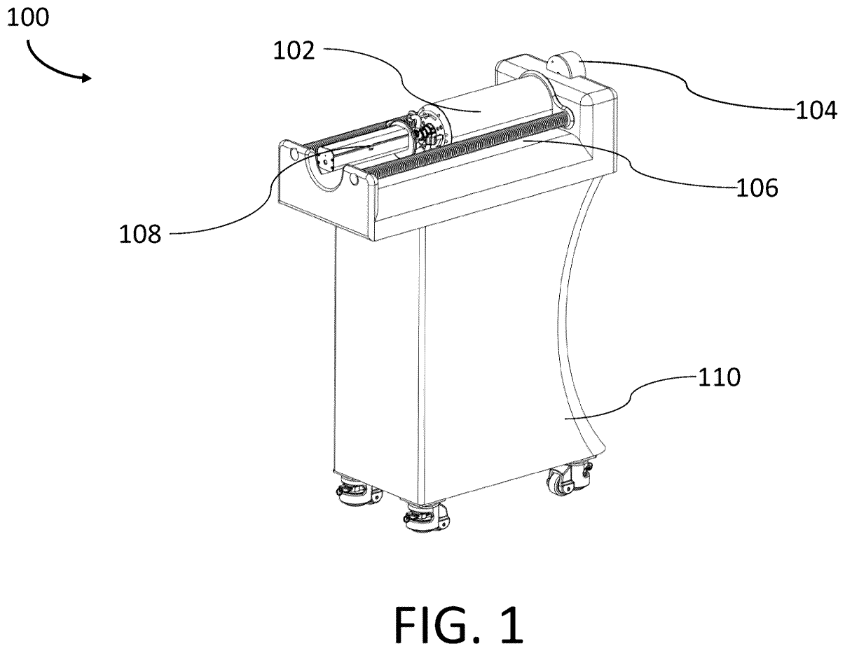

The disclosed biopsy robot may be a robotically driven needle guide with 4 or more degrees of freedom (DOF) and a 1-DOF needle driver. The biopsy robot 100 may comprise three or more main parts (FIG. 1). The first main part may be the robot cart 110, which may have a linear mechanism 106 on top to guide the robot inside the MRI scanner. The second part may be the manipulator 102 that orients the needle guide. The manipulator can be inserted into the cartridge 104 of the biopsy robot 100. The third part may be the needle driver 108 that inserts and fires the biopsy needle to collect tissue samples.

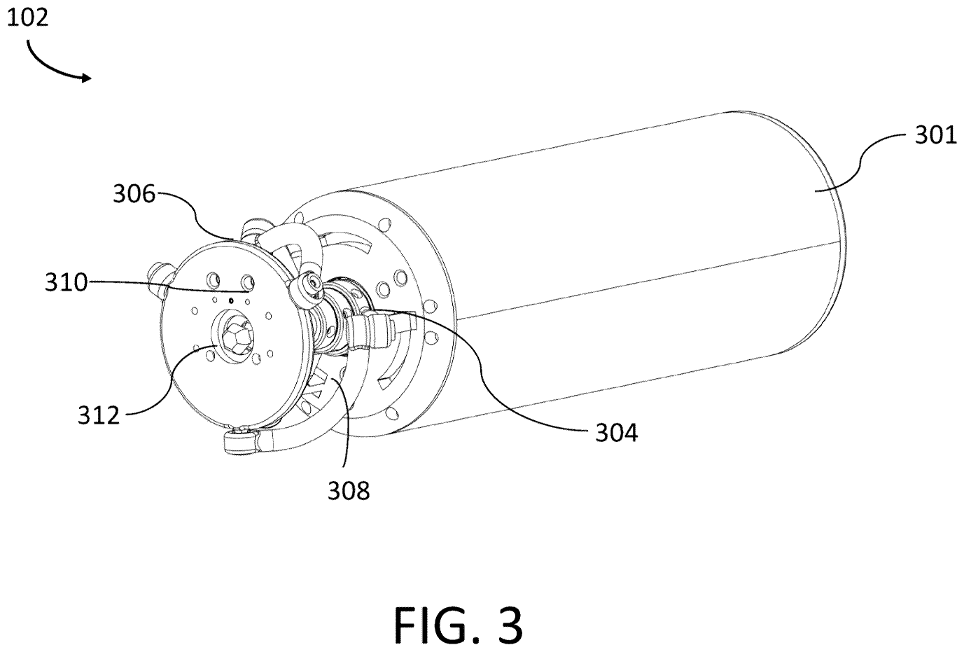

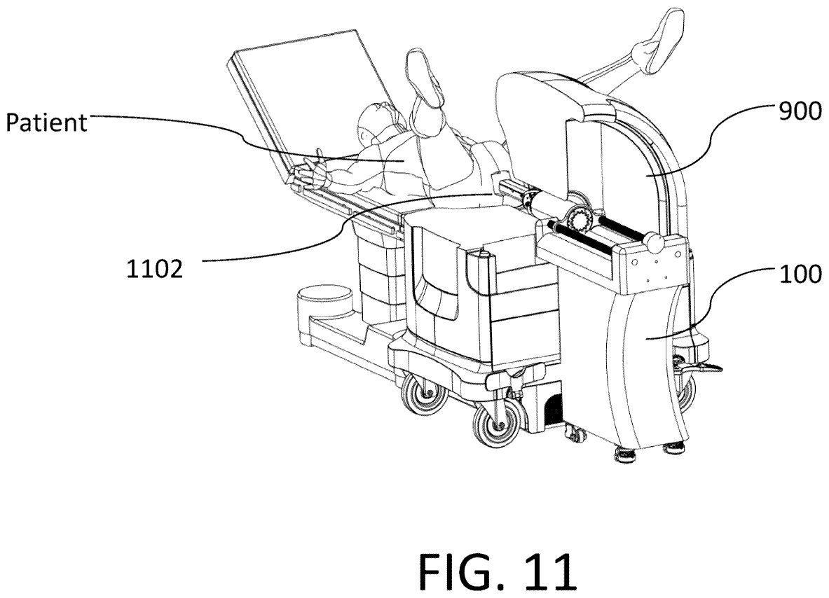

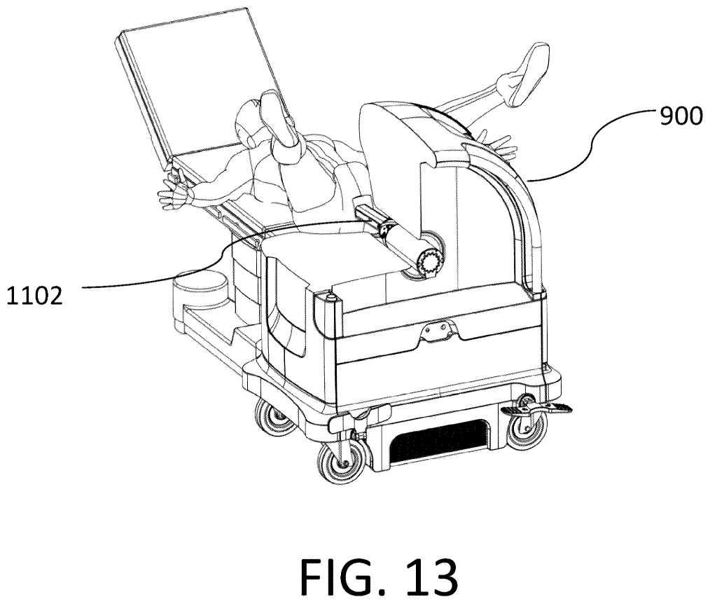

In the first configuration, the linear mechanism can drive the robot 100 inside the bore (FIG. 11). The robot cart comprising the needle and its holder 1102 can be attached and secured to the MRI scanner 900. The developed robotic system 100 may be shown for an MRI-guided prostate biopsy procedure. A part of the scanner 900 may be cropped for a better view. The robot cart can be eliminated in the second configuration, and the manipulator can be fixed inside the MRI bore (FIG. 13). The second part may be a parallel manipulator 192 with three or more degrees of freedom in which the base platform of the manipulator may be connected to the needle driver by three or more parallel identical kinematic chains 304, as shown in FIG. 3. The manipulator may comprise a base 301, three or more identical kinematic chains 304, and an end-effector 306. The end-effector 306 and manipulator 102 may be at its home position when it is parallel to the base 301. The base may be designed to fit inside the scanner bore and can match the shape of the bore. When the three or more proximal arms rotate and each move in front of one home positioning sensor 308, the end-effector 306 may be parallel to the base.

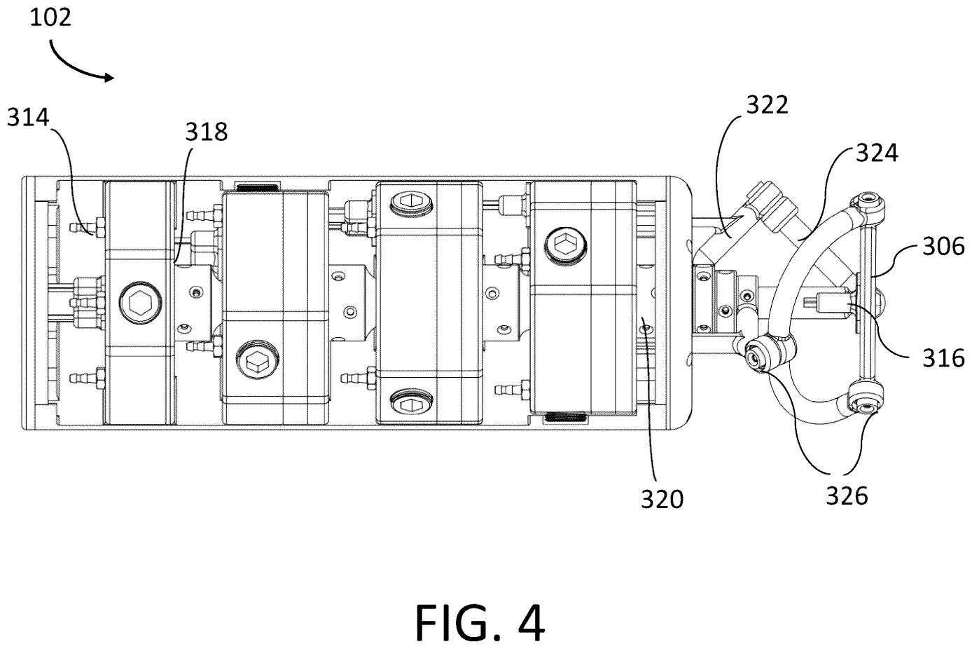

The top cover of the manipulator may be removed to have an internal view of the system. As seen in FIG. 4, on each kinematics chain, at least one fixed actuated revolute joint 320 and two or more free revolute joints 326 may connect the proximal 322 and distal arms 324 with the end-effector 306. Three or more free revolute joints 326 can connect the distal arms 324 with the end-effector 306. Three or more high-torque and resolution rotary motors can actuate the proximal arms 322 on the base. This manipulator 102 can provide three rotational DOFs: roll, pitch, and yaw.

One main characteristic feature of this parallel manipulator may be that the axes of rotation of all the joints intersect at a common point called the geometric center 310 of the manipulator (FIG. 3). The geometric center 310 may be a point at which every needle driver element rotates about that point. In other words, the location of this point may not change with respect to the manipulator base.

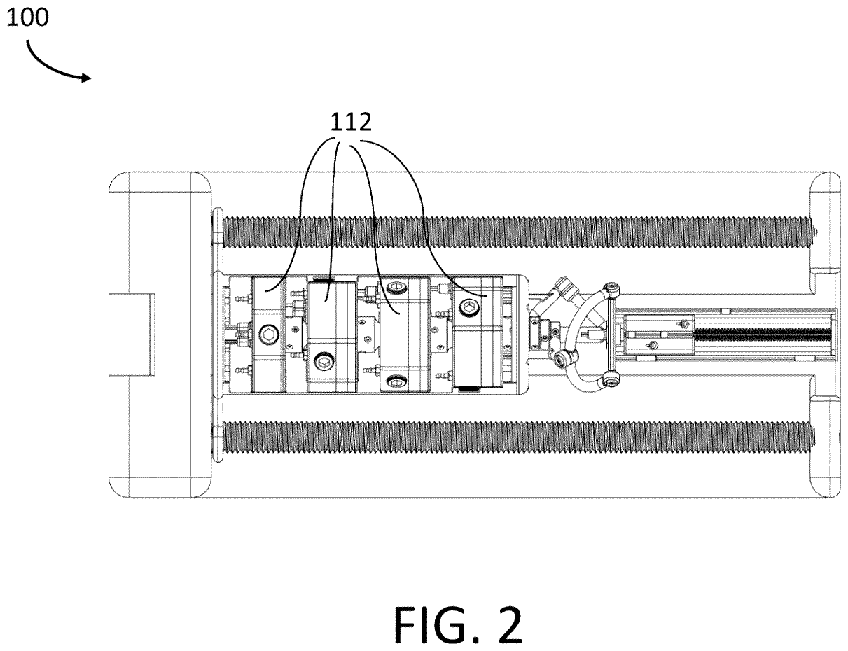

Another feature of the robot 100 may be that the manipulator base 301 contains four or more collinear actuators (FIG. 2 and FIG. 4). The top covers of the manipulator 102 and needle driver in the robotic system 100 may be removed to have an internal view of the system. Three or more high-torque and resolution pneumatic stepper motors 112 may orient the needle driver. One or more pneumatic stepper motor 112 may insert the needle into the target lesion. FIG. 4 also shows air inlet 314 that may be used for cooling the system.

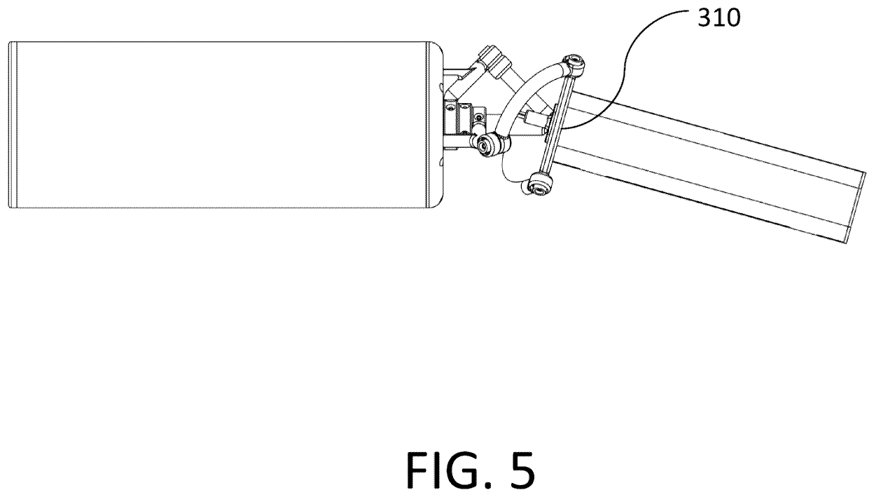

FIG. 5 illustrates a top view of a manipulator and a needle driver at a random configuration other than the home position, in accordance with some embodiments. As can be seen, many elements of the end-effector may be rotating around the geometric center 310 of the robot. The needle driver may have a lid to mitigate cross-contamination.

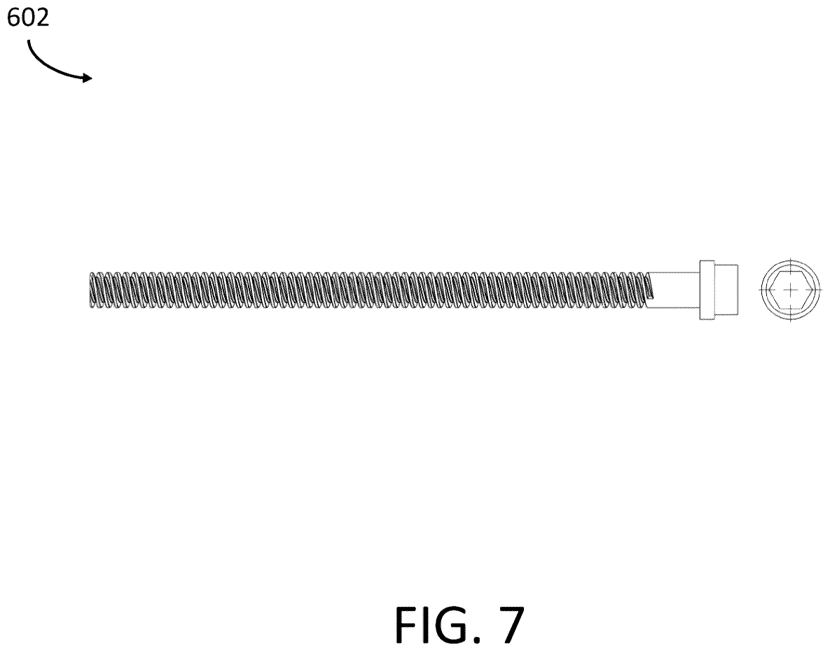

The shafts transferring the motion from actuators to kinematic chains may be designed hollow to make the collinear actuator configuration possible. The center axis of the actuators may be designed to be hollow as well. The output of the fourth actuator may be extended to the geometric center to transfer the rotational motion to the needle driver. Since the needle driver can rotate about the roll, pitch, and yaw axis, a round-head Allen mechanism 312 may be designed to transfer the rotation to the lead screw. The mechanism may comprise a cylindrical tool with a hexagonal shape at one end (FIG. 3), which fits into the socket of the lead screw 602 (FIG. 7).

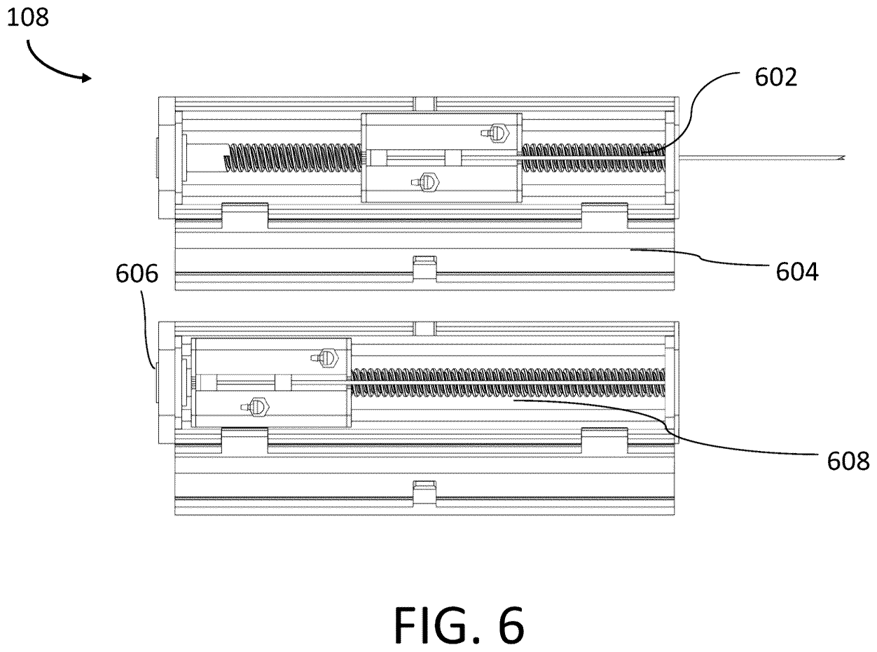

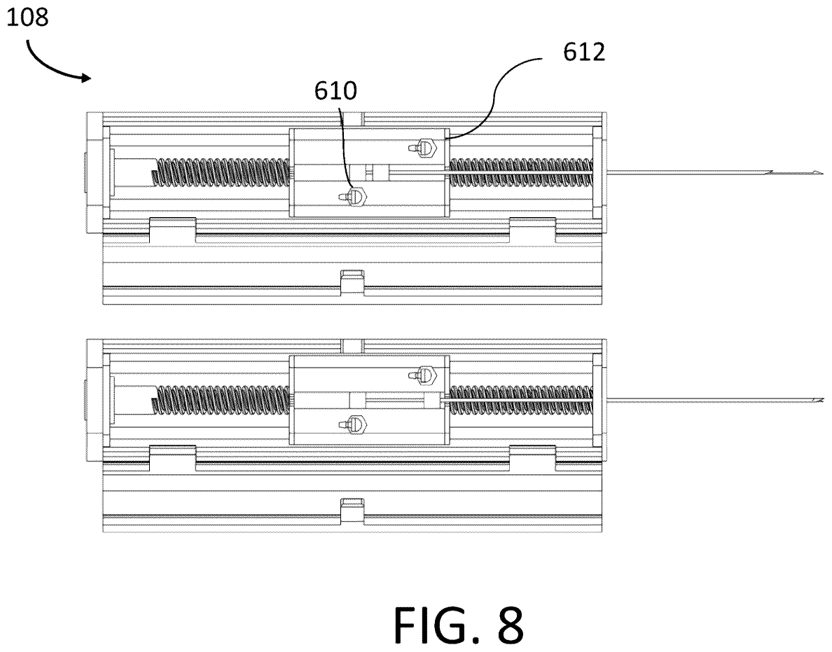

An automatic needle driver 108 may insert the biopsy needle inside the patient to the target lesion (FIG. 6 and FIG. 8). The needle driver lid 604 may be open for internal view. The needle driver may comprise a lead screw 602, a nut, and two linear pneumatic actuators. A lead screw 602 and nut mechanism may be used to drive the needle precisely. The inside of part of the needle driver 108 may be threaded to match the lead screw threads at location 608. There is also a hexagonal socket 606 to match the hexagonal head of the lead screw 602. Also, there may be an optical sensor 316 (FIG. 4) on the end-effector behind the needle driver for home positioning the needle. The lead screw 602 and nut mechanism may be a mechanism that converts rotary motion into precise linear motion. When the lead screw 602 rotates, the nut may move along the screw to move the needle forward. Two or more pneumatic actuators may be used to trigger the biopsy needle. One pneumatic actuator may be attached to the inner stylet, and a second pneumatic actuator may be connected to the cutting cannula. As shown in FIG. 8, after reaching the target lesion, the first linear pneumatic (top) may actuate the inner stylet forward then the second pneumatic actuator (bottom) can push the cutting cannula over the inner stylet to collect the sample. The needle may move back to extract the sample. There may be separate air inlets for the two pneumatic actuators. The needle driver 108 can comprise air inlets for the first linear pneumatic actuator 610 and air inlets for the second linear pneumatic actuator 612. There are two sets of optical sensors 316 (FIG. 4) that can be used to confirm accurate needle cockings and firings.

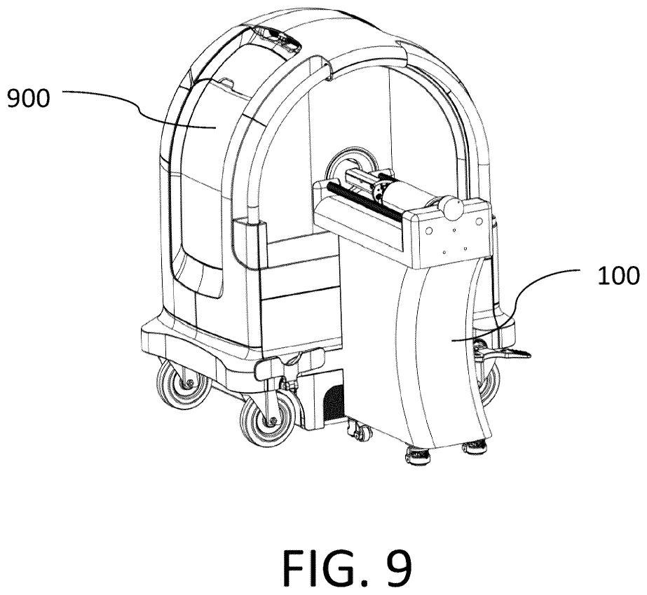

FIG. 9 shows, in an isometric view, that the robot 100 can be attached to the low-field MRI scanner 900.

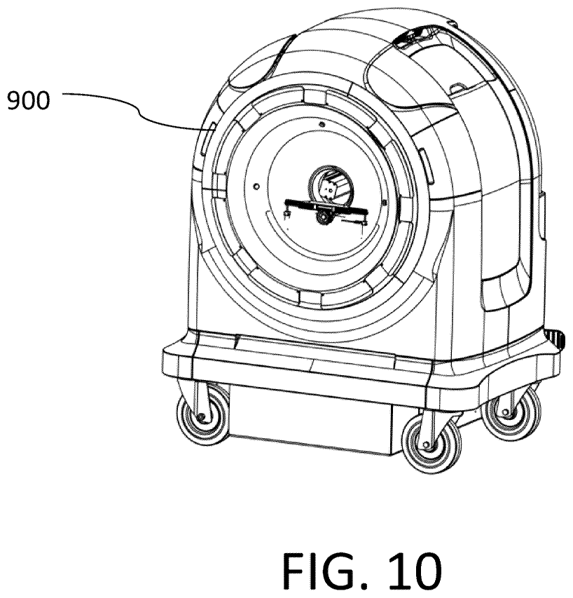

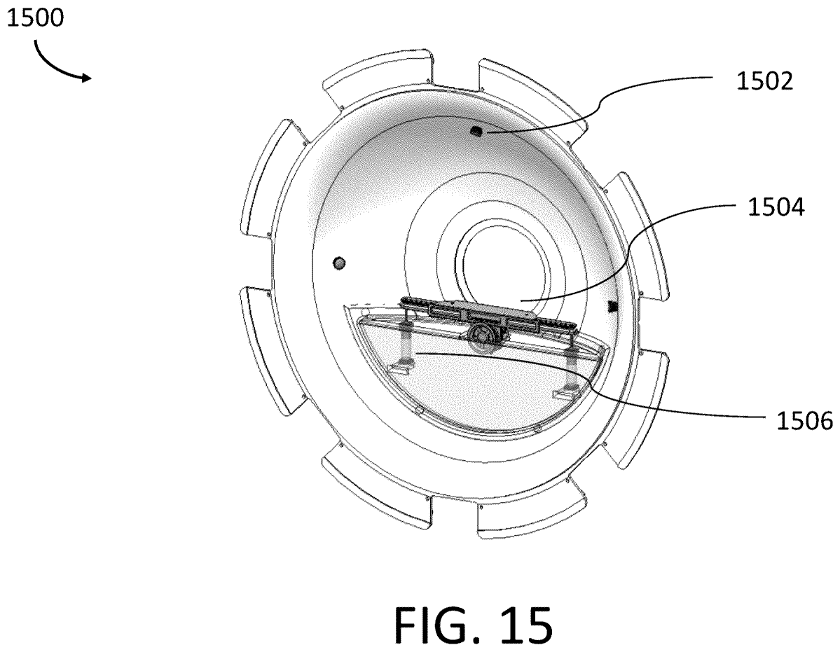

The sample tray in FIG. 10 and FIG. 15 can move in two directions horizontally to accommodate the sample location on the tray grid and in two directions vertically to adjust the height of the tray to let the sample needle deposit the tissue. The front dish is equipped with miniature cameras 1502 to enable live monitoring of patients and a sample tray.

FIG. 10 illustrates an isometric view of a manipulator and needle driver inside a low-field MRI system 900 and the sample collection mechanism in front, in accordance with some embodiments. Since the manipulator shape may be the same as the bore, it can move in the bore easily. It can also be fixed inside the bore. The sample collection has a tray on which the samples can be deposited on. The tray can move up, down, left, and/or right to get in front of the robotic arm.

FIG. 15 illustrates a stand-alone configuration of the sample collection mechanism 1500 attached to the MRI scanner dish. A precise actuator can move the deposit tray 1504 from left to right or vice versa to collect tissue sampling from the biopsy needle. The tray comprises an adjustable tray height mechanism 1506 that can move up and/or down to adjust the height of the tray. Miniature cameras 1502 can record, monitor, and provide the status of the tissue samplings.

FIGS. 11-14 may demonstrate patient location in respect to the low-field MRI as well as the robot. FIG. 11 shows an isometric view of a biopsy robot 100 comprising a needle and its holder 1102, MRI scanner 900, and a subject. FIG. 13 shows an isometric view the needle and its holder 1102 and MRI scanner 900.

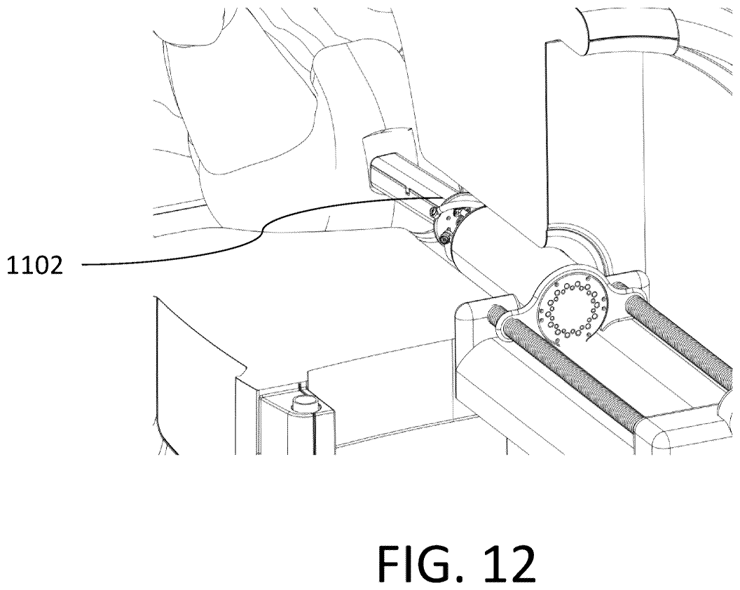

FIG. 12 illustrates a magnified isometric view of a view of an example system while operating a prostate biopsy procedure, in accordance with some embodiments. The linear actuator on the robot cart may guide the manipulator to a location close to the patient. The robot cart may comprise the needle and its holder 1102. The manipulator may orient the needle driver. Then the needle may be inserted into the target lesion. A part of the scanner may be cropped for a better view.

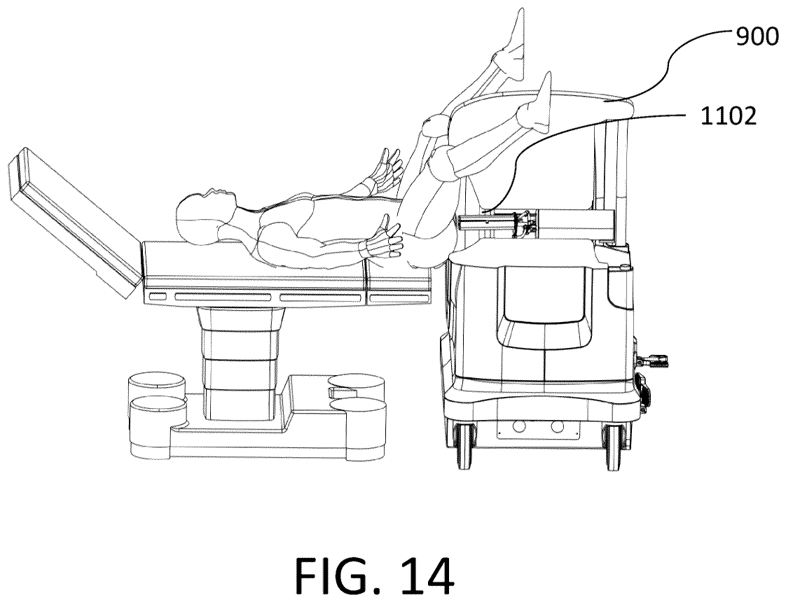

FIG. 14 illustrates side view of a stand-alone configuration of the manipulator, needle driver, needle holder 1102, and MRI scanner 900 in accordance with some embodiments. The manipulator can be fixed inside an MRI bore and operate as well. A part of the scanner 900 may be cropped for a better view.

The manipulators' base can be designed in different shapes and sizes to fit inside the scanner bore or be mounted on a patient bed. A bundle of tubes, optic fibers, pneumatic air distributors, electrical circuits, a robot controller, or any combination thereof may be used to drive the robot. This equipment may be placed far from the magnetic fields. This equipment can be designed to positioned within the MRI system environment to not interfere with the main magnetic field (BO), gradient fields, or the radio frequency (RF) fields of the MRI system, in particular within the imaging region of interest. This equipment can operate reliably within the MRI system environment and not be affected by the main magnetic field (BO), gradient fields, or the RF fields of the MRI system.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The section headings used herein are for organizational purposes only and are not to be construed as limiting the subject matter described.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

1. A biopsy robot system, comprising:
(a) a needle driver configured to insert a biopsy needle into a subject;
(b) a linear mechanism configured to place the needle driver inside a magnetic resonance scanner bore; and
(c) a parallel manipulator configured to orient or position the linear mechanism.

2. The biopsy robot system of claim 1, further comprising a robot cart configured to house components (a)-(c).

3. The biopsy robot system of claim 2, wherein the robot cart is configured to be attached, secured, or both to a MRI scanner.

4. The biopsy robot system of claim 1, further comprising at least one optical sensor configured to be placed behind the needle driver to position the needle.

5. The biopsy robot system of claim 4, wherein the at least one optical sensor is configured to confirm an accuracy of needle cockings and firings.

6. The biopsy robot system of any claim 1, wherein the parallel manipulator is fixed inside the magnetic resonance scanner bore.

7. The biopsy robot system of claim 1, wherein the parallel manipulator provides at least three degrees of freedom.

8. The biopsy robot system of claim 1, wherein the parallel manipulator comprises a base and an end effector, wherein the base is connected to the end-effector using a plurality of parallel kinematic chains.

9. The biopsy robot system of claim 1, wherein the needle driver has at least one degree of freedom.

10. The biopsy robot system of claim 1, wherein the biopsy robot system has at least four degrees of freedom.

11. The biopsy robot system of claim 1, wherein the biopsy robot system is configured for use in a fully automated biopsy procedure.

12. The biopsy robot system of claim 1, wherein the biopsy robot system comprises one or more of plastics, thermoplastic polymers, polyvinyl chloride (PVC), polyethylene terephthalate (PET), ceramics, rubbers, or any combination thereof.

13. The biopsy robot system of claim 1, wherein the biopsy robot system is configured for use with a low-field strength MRI scanner.

14. The biopsy robot system of claim 2, wherein the robot cart comprises the linear mechanism.

15. The biopsy robot system of claim 8, wherein at least some chains of the plurality of parallel kinematic chains comprise at least one fixed revolute joint and a plurality of free revolute joints.

16. The biopsy robot system of claim 15, wherein the at least one fixed revolute joint and the plurality of free revolute joints are configured to couple proximal arms and distal arms of the end-effector.

17. The biopsy robot system of claim 16, further comprising a plurality of rotary motors configure to actuate proximal arms coupled to the base.

18. The biopsy robot system of claim 16, further comprising a plurality of free revolute joints configured to couple distal arms to the end-effector.

19. The biopsy robot system of claim 1, wherein the parallel manipulator has a geometric center around which every needle driver element is configured to rotate.

20. The biopsy robot system of claim 1, wherein the needle driver comprises an automatic needle driver.
